(19) United States
(12) Patent Application Publication
(10) Pub. No.: US 2009/0270470 A1
(43) Pub. Date: Oct. 29, 2009

(54) Compounds I

(75) Inventors: Catrin Jonasson, Sodertalje (SE); Hakan Molin, Sodertalje (SE); Berit Molin, Sodertalje (SE); Didier Rotticci, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/912,551

(22) PCT Filed: May 8, 2006
(86) PCT No.: PCT/SE2006/000548

§ 371 Date: Oct. 25, 2007

(30) Foreign Application Priority Data
May 9, 2005 (SE) 0501057-4

(51) Int. Cl. A61K 31/41; C07C 255/00; A61K 31/15; C07D 257/04; C07C 233/00; A61K 31/165

(52) U.S. Cl. 514/381; 558/303; 514/664; 548/250; 564/183; 514/617

(57) ABSTRACT

Compounds of formula I,

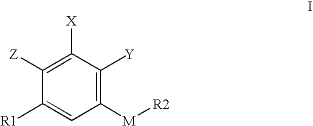

wherein R1, Z, X, Y, M and R2 are as defined in the specification, pharmaceutically acceptable salts thereof, pharmaceutical composition containing the same, methods of using the same for therapeutic purposes and methods of making the same.

### FIELD OF THE INVENTION

The present invention relates to new compounds of formula I, as a free acid and/or base or a pharmaceutically acceptable salt, solvate or solvate of salt thereof. The present invention also relates to use of such compounds in therapy, and also pharmaceutical formulations containing such compounds. The present invention further relates to a process for the preparation of compounds of formula I.

### BACKGROUND OF THE INVENTION

The inhibitory glycine receptors (GlyRs) are ion channels belonging to the cys-loop ligand-gated ion channel family. They are pentameric structures composed of two types of membrane spanning subunits (α and β ) forming a pore that is permeable to anions. The sub-units have four transmembrane domains and a large extracellular N-terminus.

Four distinct α subunits (α1(Pfeiffer, F, H Betz. Brain Research 226, 273-9. 1981); (Pfeiffer et al Journal of Biological Chemistry 257, 9389-93. 1982), α2 (Becker et al EMBO Journal 7, 3717-26. 1988); (Akagi, H, K Hirai, F Hishinuma. FEBS Letters. 281, 160-6. 1991; Kuhse, J, V Schmieden, H Betz, 1990a, Neuron, v. 5, p. 867-73), α3 (Kuhse, J, V Schmieden, H Betz, 1990b, J Biol Chem, v. 265, p. 22317-20) α4 (Harvey, et al, European Journal of Neuroscience 12, 994-1001. 2000)) and one β subunit (Pfeiffer and Betz, 1981), (Pfeiffer et al., 1982) have been identified. All subunits except α4 do appear to exist in humans. The predominant receptor isoform consists of α1-and β-subunits with a possible stoichiometry 3α2β. In recombinant systems, homo-oligomeric α-subunits (homomeric GlyR α1) function efficiently with functional properties similar to those of native receptors.

GlyRs are located at postsynaptic membranes mainly in the spinal cord and brain stem (Rajendra, S, J W Lynch, P R Schofield. Pharmacology & Therapeutics 73, 121-46. 1997); (Laube, B, G Maksay, R Schemm, H Betz. Trends in Pharmacological Sciences 23, 519-527. 2002). Glycinergic neurons in the dorsal horn receive a major input from myelinated low-threshold mechanoreceptive primary (Aβ) afferents. Binding of an agonist induces rapid opening of the channel and allowing an influx of Cl− into the cytoplasm. The following hyperpolarisation of the postsynaptic membrane stabilises the resting potential of the cell and thus inhibits neuronal firing. It has been suggested that loss of this inhibitory modulation, as might occur following peripheral or central nerve injury, could facilitate synaptic connections between Aβ-fibers and pain-signalling pathways, thereby resulting in the miscoding of this input as pain. This has been modelled experimentally in animals by the spinal administration of the specific glycine receptor antagonist strychnine (Sorkin, L S, S Puig. Pain 68, 283-92. 1996); (Sherman, S E, C W Loomis. Pain 56, 17-29. 1994); (Sherman, S E, C W Loomis. Canadian Journal of Physiology & Pharmacology 73, 1698-705. 1995; Sherman, S E, C W Loomis. Pain 66, 321-330. 1996); (Yaksh, T L, 1989, Pain, v. 37, p. 111-23); (Beyer, C, C Banas, P Gomora, B R Komisaruk. Pharmacology, Biochemistry & Behavior 29, 73-8. 1988); (Onaka, M, T Minami, I Nishihara, S Ito. Anesthesiology 84, 1215-22. 1996);

Further, it has been shown that mice deficient in GlyR α3 show a reduction in pain sensitisation induced by spinal PGE2 injection or peripheral inflammation. GlyR α3 deficient mice do also lack PGE2 induced inhibition of glycinergic neurotransmission (Harvey, R J, U B Depner, H Wassle, S Ahmadi, C Heindl, H Reinold, T G Smart, K Harvey, B Schutz, O M Abo-Salem, A Zimmer, P Poisbeau, H Welzl, D P Wolfer, H Betz, H U Zeilhofer, U Muller. Science 304, 884-887. 2004).

Positive modulators or agonists of GlyR could be therapeutically beneficial in all conditions with impaired inhibitory tone, specifically as analgesics in neuropathic or inflammatory pain syndromes, such as painful diabetic neuropathy, post traumatic neuralgia, post herpetic neuralgia, trigeminal neuralgia, arthritis, rheumatoid diseases, fibromyalgia, low back pain with radiculopathy and post-operative pain. Further in pain associated with various conditions including angina, renal or billiary colic, menstruation, migraine and gout, stroke, head trauma, anoxic and ischemic injuries, hypoglycaemia, cardiovascular diseases and cancer. GlyR agonists or positive modulators could also be used as anticonvulsants and muscle-relaxants as well as anti-inflammatory agents.

Glycine receptors are also involved in the acrosome reaction (AR) and activation of GlyRs seems to be essential for the AR to occur. GlyR agonists or positive modulators could therefore be useful as fertility enhancers or as a male contraceptive. Glycine receptors are also expressed in the auditory pathways and in the retina. GlyR positive modulators or agonists could therefore be used in the treatment of auditory neuropathic disorders such as tinnitus and ophthalmological disorders such as retinopathies, diabetic retinopathies and glaucoma (Lynch, J W. Physiol. Rev. 84, 1051-1095. 2004).

Glycine receptor subunits have also been identified in the nucleus accumbens and GlyR selective compounds have been suggested to combat psychiatric disorders, in which the mesolimbic dopamine system is implicated, such as alcoholism, drug addiction and psychosis (Molander, A, B Söderpalm. Alcoholism: Clinical and Experimental Research 29, 17-26. 2005).

Prostaglandins and leukotrienes are produced by the activity of three enzymes; cyclooxygenase-1, cyclooxygenase-2 (COX-1 and COX-2) and 5-lipoxygenase (5-LOX), as part of the arachidonic acid (AA) pathway. COX-1 converts AA to e.g. prostaglandins such as PGD2, PGE2, PGF2 and PGI2 (prostacyclin) and thromboxanes such as TXA2. COX-2 converts AA to a narrower range of prostaglandins, specifically PGE2 and PGI2. 5-LOX together with other enzymes converts AA to leukotrienes (LTB4, LTC4, LTD4 and LTE4). The products from the AA pathway play a major role in human physiology that includes renal homeostasis, gastroprotection, vascular homeostasis and pathophysiological processes, such as pain and inflammation.

PGE2 and PGI2 have various physiological and pathophysiological effects. For example they have potent effects on vasodilatation and vascular permeability.

Inhibitors of cyclooxygenases have been developed as anti-inflammatory drugs as have inhibitors of 5-lipoxygenase. Dual COX/LOX inhibitors are in the clinic for evaluation of inflammation related diseases, such as rheumatoid arthritis and osteoarthritis as well as pneumological diseases. They could also be used in arthrosclerosis and stroke. Further they could be used as antihypertensive agents (Simmons, D L, Botting Regina M., T Hla. Pharmacol Rev 56, 387-487. 2004), (Bertolini, A, A Ottani, Sandrini M. Current Medicinal Chemistry 9, 1033-1043. 2002).

### SUMMARY OF THE INVENTION

The object of the present invention is thus to provide new positive modulators and/or agonists of GlyR, that are optionally also COX and/or LOX inhibitors.

Accordingly, the present invention provides compounds of formula I, or pharmaceutically acceptable salts thereof

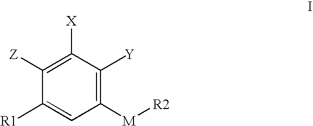

wherein

X is selected from hydrogen, halo, —CN, —CONH₂, —CON(C₁₋₆alkyl)H, —CON(C₁₋₆alkyl)₂ and heterocyclic groups;

R1 is selected from C₁₋₆alkyl and C₃₋₆cycloalkyl;

whereby
Z is —OH,
M is selected from —C(O)—, —CH(OR^{a})—, —N(R^{a})—, and —S(O)ᵣ—, wherein R^{a} is hydrogen or C₁₋₆alkyl and r is 0, 1 or 2,
R2 is selected from C₁₋₆alkyl, C₃₋₆cycloalkyl, heterocycloalkyl, aryl, alkylaryl, and heteroaryl,
R2 is substituted with halo, —NO₂, —CN, —OH, —CF₃, —OCF₃, —NH₂, and/or —CONH₂, when Y is —C₁₋₆alkyl; or
Z is —C₁₋₆alkoxy,
M is bond,
R2 is selected from C₁₋₆alkyl, C₃₋₆cycloalkyl, heterocycloalkyl, aryl, alkylaryl, and heteroaryl,
when Y is —C₁₋₆alkoxy.

In another aspect of the invention there is provided the compound of formula I, or pharmaceutically acceptable salts thereof,

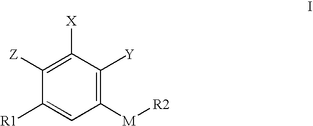

wherein

X is selected from hydrogen, halo, —CN, —CONH₂, —CON(C₁₋₆alkyl)H, —CON(C₁₋₆alkyl)₂ and heterocyclic groups;

R1 is selected from C₁₋₆alkyl and C₃₋₆cycloalkyl;

whereby
Z is —OH,
M is selected from —C(O)—, —CH(OR^{a})—, —N(R^{a})—, and —S(O)ᵣ—, wherein R^{a} is hydrogen or C₁₋₆alkyl and r is 0, 1 or 2,
R2 is selected from C₁₋₆alkyl, C₃₋₆cycloalkyl, heterocycloalkyl, aryl, alkylaryl, and heteroaryl,
R2 is substituted with halo, —NO₂, —CN, —OH, —CF₃, —OCF₃, —NH₂, and/or —CONH₂, when Y is —C₁₋₆alkyl; or
Z is —C₁₋₆alkoxy,
M is bond,
R2 is selected from C₁₋₆alkyl, C₃₋₆cycloalkyl, heterocycloalkyl, aryl, alkylaryl, and heteroaryl,
when Y is —C₁₋₆alkoxy,

for use in therapy.

In a further aspect of the invention there is provided the compound of formula I, or pharmaceutically acceptable salts thereof, for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

In a further aspect of the invention there is provided a pharmaceutical composition comprising a therapeutically effective amount of the compound of formula I in association with one or more pharmaceutically acceptable diluent, excipients and/or inert carrier, especially for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular is diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

Another aspect of the invention relates to the use of the compound according formula I in the manufacture of a medicament for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

In a further aspect of the invention there is provided a method of treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis, comprising administering to a mammal, including man, in need of such treatment, a therapeutically effective amount of the compound according formula I.

In yet another aspect of the invention there is provided processes for the preparation of compounds of formula I.

These and other aspects of the present invention are described in greater detail herein below.

### DETAILED DESCRIPTION OF THE INVENTION

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

For the avoidance of doubt it is to be understood that in this specification ‘C₁₋₆’ means a carbon group having 1, 2, 3, 4, 5 or 6 carbon atoms.

In the case where a subscript is the integer 0 (zero) the group to which the subscript refers to indicates that the group is absent.

In this specification unless otherwise stated the term “heteroatom” refers to an atom which is not carbon or hydrogen. Examples of heteroatoms include but are not limited to nitrogen, oxygen, and sulfur.

In this specification, unless stated otherwise, the term “alkyl” includes both straight and branched chain alkyl groups. The term “C₁₋₆alkyl” means an alkyl group having 1 to 6 carbon atoms and may be methyl, ethyl, n-propyl, i-propyl, n-butyl, i-butyl, s-butyl, t-butyl, n-pentyl, i-pentyl, t-pentyl, neo-pentyl, n-hexyl, i-hexyl, or t-hexyl.

In this specification, unless stated otherwise, the term “alkoxy” includes both straight or branched alkoxy groups. C₁₋₆alkoxy may be, but is not limited to, methoxy, ethoxy, n-propoxy, i-propoxy, n-butoxy, i-butoxy, s-butoxy, t-butoxy, n-pentoxy, i-pentoxy, t-pentoxy, neo-pentoxy, n-hexoxy, i-hexoxy, or t-hexoxy.

In this specification, unless stated otherwise, the term “halo” and “halogen” may be fluoro, chloro, bromo, or iodo.

In this specification, unless stated otherwise, the term “aryl” includes both aromatic monocyclic and bicyclic systems containing from 5 to 10 carbon atoms; in the case of a bicyclic system, at least one of the rings is of aromatic character, while the other ring may be aromatic or partially hydrogenated. Non-limiting examples of the term “aryl” are phenyl, naphthyl, indenyl, and tetralinyl.

In this specification, unless stated otherwise, the term “alkylaryl” means an aryl group having one or more alkyl groups pendant therefrom. Non-limiting examples of the term “alkylaryl” are benzyl, ethylnaphthyl, propylindenyl, and butyltetralinyl

In this specification, unless stated otherwise, the term “heteroaryl” includes aryl groups as described above in which 1 to 4 carbon atoms are replaced by 1 to 4 hetero atoms, identical or different, selected independently of each other from oxygen, sulfur and nitrogen. Non-limiting examples of the term “heteroaryl” are furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyl.

In this specification, unless stated otherwise, the term “cycloalkyl” includes both monocyclic and polycyclic systems containing from 3 to 10 carbon atoms, the systems being saturated or partially unsaturated but without aromatic character and it being understood that in the case of a polycyclic system one or more of the cycle(s) could be fused together or form a link. By the term “C₃₋₆cycloalkyl” is meant a cycloalkyl group containing from 3 to 6 carbon atoms, and may be cyclopropyl, cyclobutyl, cyclopentyl, or cyclohexyl. In this specification, unless stated otherwise, a “heterocyclic group” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxide(s). Non-limiting examples of the term “heterocyclic group” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, 4-pyridone, 1-isoquinolone, 2-pyrrolidone and 4-thiazolidone.

In this specification, unless stated otherwise, the term “heterocycloalkyl” includes cycloalkyl groups as defined hereinbefore in which 1 to 4 carbon atoms are replaced by 1 to 4 heteroatoms. Non-limiting examples of the term “heterocycloalkyl” are tetrahydrofuran, tetrahydrothiophene, piperidine, piperazine, morpholine, thiomorpholine, tetrahydropyran, tetrahydrothiopyran.

One aspect of the invention relates to compounds of formula I, wherein X may be independently selected from hydrogen, halo, —CN, —CONH₂, and heterocyclic groups. In a specific aspect X may be independently selected from hydrogen, —Br, —CN, —CONH₂, and tetrazolyl.

According to one aspect of the invention, R1 is C₃₋₄alkyl.

According to one aspect of the invention Z is —OH, M is —C(O)—, R2 is selected from C₁₋₆alkyl, C₃₋₆cycloalkyl, heterocycloalkyl, aryl, alkylaryl, and heteroaryl, R2 is substituted with halo, —NO₂, —CN, —OH, —CF₃, —OCF₃, —NH₂, and/or —CONH₂, and Y is —C₁₋₆alkyl.

One aspect of the invention relates to compounds of formula I, wherein R2 is aryl. In a specific aspect R2 may be independently selected from phenyl, naphthyl, cyclohexyl, methylbenzyl, and quinoxalinyl.

In one aspect of the invention Z is —OH, M is selected from —C(O)—, —CH(OR^{a})—, —N(R^{a})—, and —S(O)ᵣ—, wherein R^{a} is hydrogen or C₁₋₆alkyl and r is 0, 1 or 2, R2 is selected from C₁₋₆alkyl, C₃₋₆cycloalkyl, heterocycloalkyl, aryl, alkylaryl, and heteroaryl, R2 is substituted with halo, particularly chloro, and Y is —C₁₋₆alkyl.

In one aspect of the invention X is selected from hydrogen, —Br, —CN, —CONH₂, and tetrazolyl; Y is —CH₃; R1 is C₃₋₄alkyl; M is —C(O)—; R2 is selected from phenyl, naphthyl, cyclohexyl, methylbenzyl, and quinoxalinyl; R2 is substituted with chloro; and Z is —OH.

In one aspect of the invention X is selected from hydrogen, —Br, —CN, —CONH₂, and tetrazolyl; Y is —OCH₃; R1 is C₃₋₄alkyl; M is a bond; R2 is selected from phenyl, naphthyl, cyclohexyl, methylbenzyl, and quinoxalinyl; and

Z is —OCH₃.

In yet another aspect of the invention, there is provided compounds, said compounds being:
(4-chlorophenyl) [4-hydroxy-5-isopropyl-2-methyl-3-(1H-tetrazol-5-yl)phenyl]methanone,
(4-hydroxy-5-isopropyl-2-methylphenyl)(quinoxalin-2-yl)methanone,
3-(4-chlorobenzoyl)-6-hydroxy-5-isopropyl-2-methylbenzamide,
3-(4-chlorobenzoyl)-6-hydroxy-5-isopropyl-2-methylbenzonitrile,
3,5-di-tert-butyl-2,6-dimethoxybenzamide, and
1,5-di-tert-butyl-2,4-dimethoxybenzene

A suitable pharmaceutically acceptable salt of the compound of the invention is, for example, an acid-addition salt, for example an inorganic or organic acid. In addition a suitable pharmaceutically acceptable salt of the compounds of the invention is an alkali metal salt, an alkaline earth metal salt or a salt with an organic base that affords a physiologically-acceptable cation.

Some compounds of formula I may have chiral centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

### Pharmaceutical Composition

According to one aspect of the present invention there is provided a pharmaceutical composition comprising as active ingredient a therapeutically effective amount of the compound of formula I, or salts, solvates or solvated salts thereof, in association with one or more pharmaceutically acceptable diluent, excipients and/or inert carrier.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers.

Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, severity of the illness being treated, the route of administration, is the age, weight and sex of the patient and the particular compound being used, and may be determined by a physician.

### Medical Use

The compounds of the present invention are expected to be useful in the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

The invention relates to compounds of formula I as defined hereinbefore, for use in therapy.

The invention relates to compounds of formula I as defined hereinbefore, for use in treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

The present invention relates also to the use of a compound of formula I as defined hereinbefore, in the manufacture of a medicament for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

One embodiment of the invention relates to the use of a compound according to formula I in the treatment of acute and chronic neuropathic pain.

The invention also provides a method of treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

Another embodiment of the invention relates to the use of a compound according to formula I, for the manufacture of a medicament for acute and chronic neuropathic pain

The dose required for the therapeutic or preventive treatment of a particular disorder will necessarily be varied depending on the host treated, the route of administration and the severity of the illness being treated.

In the context of the present specification, the term “therapy” and “treatment” includes prevention and/or prophylaxis, unless there are specific indications to the contrary. The terms “therapeutic” and “therapeutically” should be construed accordingly.

### Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; ophthalmological disorders such as retinopathies, diabetic retinopathies or glaucoma; and/or psychiatric disorders, such as alcoholism, drug addiction and psychosis.

### Methods of Preparation

Another aspect of the present invention provides processes for preparing compounds of formula I, or salts, solvates or solvated salts thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

### Preparation of End Products

A process for preparing a compound of formula I, wherein X, Y, Z, R1, and R2 are, unless specified otherwise, defined as in formula I, comprises of:

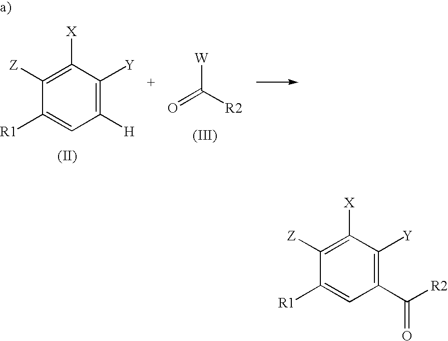

i) Reaction of a compound of formula (II) with a compound of formula (III), wherein W is a halogen, such as for example Cl, Br or F, or a suitable leaving group, such as for example trifluoromethanesulfonyloxy, 4-toluenesulfonyloxy, alkylcarbonyloxy or hydroxy.

The reaction is performed in a suitable solvent such as dichloromethane, dichloroethane, nitromethane, and advantageously in the presence of a Lewis acid such as for example AlCl₃, AlBr₃, Al(OR)₃, BF₃, BCl₃, BBr₃, ZnCl₂, FeCl₃, FeBr₃;

or

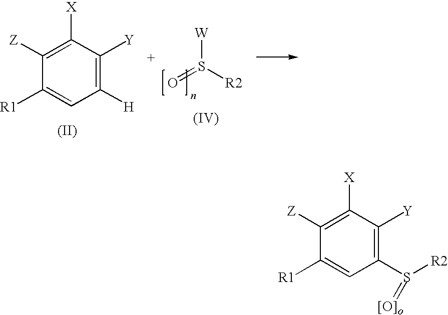

ii) Reaction of a compound of formula (II) with a compound of formula (IV), wherein n is 0, 1 or 2, o is 0, 1 or 2, and W is a halogen such as for example Cl, Br or F. When n=0 or 1 the product from the first step can be oxidized by treatment with an oxidation reagent such as for example m-Chloroperbenzoic acid, hydrogen peroxide, NaIO₄, KMnO₄, PhICl₂ or t-BuOCl.

The reaction is performed in a suitable solvent such as dichloromethane, dichloroethane, THF, DMF, optionally in the presences of a Lewis acid such as for example AlCl₃, AlBr₃, Al(OR)₃, BF₃, BCl₃, BBr₃, ZnCl₂, FeCl₃, or FeBr₃, and when n=0, advantageously in the presence of a base such as for example pyridine, lutidine, triethylamine or Hünig's base at temperatures between −10° C. to reflux.

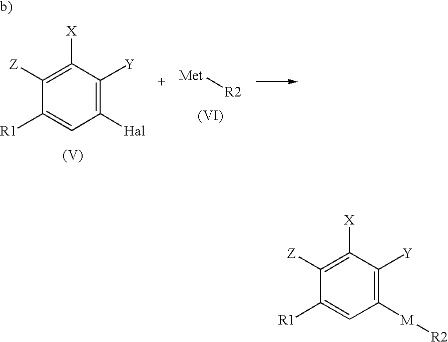

Reaction of a compound of formula (V) with an organometallic reagent of formula (VI), wherein Hal is a halogen for example Br or I; or a sulfonyloxy group, for example methanesulfonyloxy, 4-toluenesulfonyloxy, or trifluoromethanesulfonyloxy, X is a non-protic or protected functional group, for example CN, and Met is a suitable metallic group, for example, copper, lithium, an organoboron reagent such as —B(OH)₂, —B(OPri)₂ or —B(Et)₂, in the presence of a carbon monoxide or dry nitrogen atmosphere, and in the presence of a metallic catalyst such as palladium or nickel, for example [1,1′-bis(diphenylphosphino)ferrocene]dichloropalladium(II), tetrakis(triphenylphosphine)-palladium(0), palladium(II)chloride, palladium(II) bromide, nickel(II)chloride, nickel(II)bromide or bis(triphenylphosphine)nickel(II) chloride, and optionally in the presence of an additional ligand such as for example di-tert-butylphosphino pentaphenylferrocene or 2-dicyclohexylphosphino-2′,6′-dimethoxybiphenyl in the presence of a suitable inert solvent or diluent, for example tetrahydrofuran, 1,4-dioxan, 1,2-dimethoxyethane, benzene, toluene, xylene, anisol, methanol or ethanol. When Hal is Br, potassium iodide can preferably be used as an additive. The reaction is preferably conducted in the presence of a suitable base such as, for example, sodium carbonate or potassium carbonate, potassium fluoride, potassium phosphate, pyridine, 4-dimethylamino-pyridine, triethylamine or morpholine, and conveniently at a temperature in the range, for example 10 to 250° C., preferably in the range 60 to 120° C. Performing the reaction in the presence of carbon monoxide gives compounds where M is a carbonyl group, whereas performing the reaction in the absence of carbon monoxide gives compounds where M is a single bond.

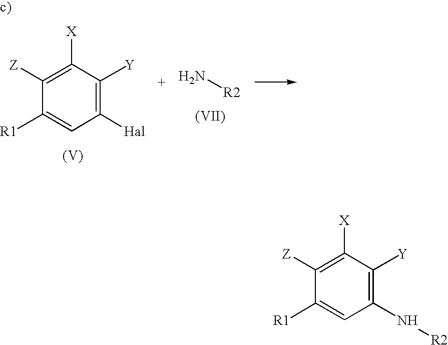

Reaction of an optionally protected compound of formula (V) with an amine of formula (VII), wherein Hal is a halogen for example Br or I; or a sulfonyloxy group, for example methanesulfonyloxy 4-toluenesulfonyloxy or trifuoromethanesulfonyloxy, X is a non-protic or protected functional group, for example CN, and in the presence of a metallic catalyst such as palladium or nickel, for example bis(dibenzylideneacetone)platinum(0), [1,1′-bis(diphenylphosphino)ferrocene]dichloropalladium(II), tetrakis(triphenylphosphine)-palladium(0), palladium(II) chloride, palladium(II) bromide, nickel(II) chloride, nickel(II) bromide or bis(triphenylphosphine)nickel(II) chloride, and optionally in the presence of an additional ligand such as for example di-tert-butylphosphino pentaphenylferrocene or 2-dicyclohexylphosphino-2′,6′-dimethoxybiphenyl in the presence of a suitable inert solvent or diluent, for example tetrahydrofuran, 1,4-dioxan, 1,2-dimethoxyethane, benzene, toluene, xylene, anisol, methanol or ethanol. When Hal is Br, potassium iodide can optionally be used as an additive. The reaction is preferably conducted in the presence of a suitable base such as, for example, sodium carbonate or potassium carbonate, potassium fluoride, potassium phosphate, pyridine, 4-dimethylaminopyridine, triethylamine or morpholine, and conveniently at a temperature in the range, for example 10 to 250° C., preferably in the range 60 to 120° C.

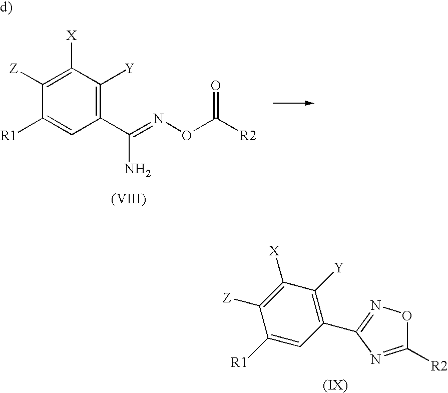

Reaction of a compound of formula (VIII), by heating in a suitable solvent for example DMF, DMA, DMSO, NMP, DMPU, toluene, xylene, tetrachloroethane at temperatures between 30° C. and reflux, to give a compound of formula (IX).

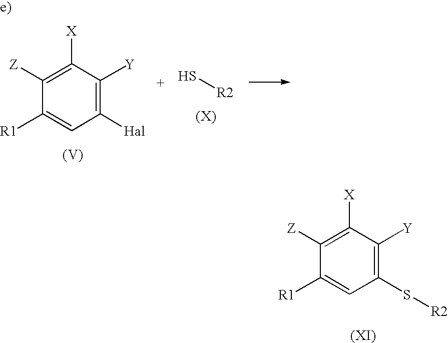

Reaction of an optionally protected compound of formula (V) with a mercaptan of formula (X), wherein Hal is a halogen for example Br or I; or a sulfonyloxy group, for example methanesulfonyloxy 4-toluenesulfonyloxy or trifluoromethanesulfonyloxy, X is a non-protic or protected functional group, for example CN, and Prot is hydrogen or a suitable protecting group, for example methyl or benzyl; in the presence of a metallic catalyst such as Cu(I) derivatives such as for example CuCl, CuBr, CuI, Cu(OCF₃) and in the presence of a suitable base such as an alkaline carbonate for example sodium carbonate, potassium carbonate or cesium carbonate in a suitable solvent or solvent mixture, for example mixtures of C₁₋₆diols and C₁₋₆alcohols such as ethylene or propylene glycol and 1-propanol, 2-propanol or tert-butanol by heating in an inert atmosphere at temperatures between 30° C. and reflux, to give a compound of formula (XI).

The product from the first step can then optionally be oxidized by treatment with an oxidation reagent such as for example m-Chloroperbenzoic acid, hydrogen peroxide, NaIO₄, KMnO₄, PhICl₂ or t-BuOCl to give a sulfoxide or sulfone.

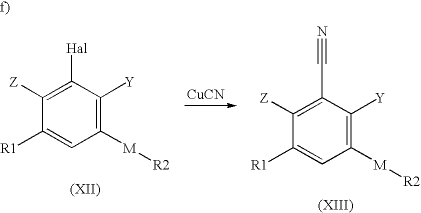

Reaction of an optionally protected compound of formula (XII) with a suitable cyanide nucleophile such as for example CuCN, wherein Hal is a halogen for example Br or I; or a sulfonyloxy group, for example methanesulfonyloxy 4-toluenesulfonyloxy or trifluoromethanesulfonyloxy, in a suitable solvent such as for example DMF, DMA, NMP or DMSO at temperatures between 50° C. and reflux under an inert atmosphere to give a compound of formula (XIII).

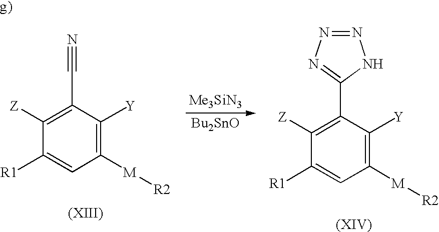

Reaction of an optionally protected compound of formula (XIII) with a suitable azide reagent such as for example trimethylsilyl azide, in the presence of a catalyst such as for example n-dibutyltin oxide in a suitable solvent such as for example toluene or xylene at temperatures between 50° C. and reflux under an inert atmosphere to give a compound of formula (XIV).

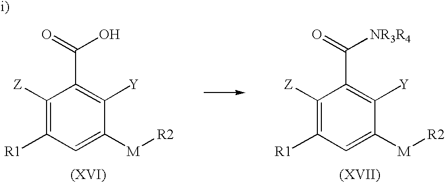

Reaction of a compound of formula (XVI), where M, R1, R2, Y and Z are as defined in compound (I) above, R3 and R4 are hydrogen, C₁₋₆alkyl, with a suitable halogenation reagent such as for example thionyl chloride, thionyl bromide, phosphoryl chloride or oxalyl chloride in a suitable solvent such as for example toluene or dichloromethane advantageously containing a small amount of a catalyst such as for example DMF at temperatures between ambient and reflux followed by treatment with amines or ammonia solution in for example water, methanol, diethyl ether to give a compound of formula (XVII).

### Preparation of Intermediates

Reaction of an optionally protected compound of formula (XV)

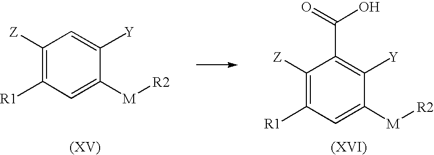

with a suitable base such as for example n-Butyllithium, sodium metal, sodium-, potassium- or cesium carbonate, sodium-, potassium- or cesium hydrogen carbonate or sodium-, potassium- or cesium hydroxide and carbon dioxide, in a suitable solvent such as for example hexane, pentane, DMF, DMA, NMP or pyridine at temperatures between −78° C. and reflux, optionally in an inert atmosphere to give a compound of formula (XVI).

### EXAMPLES

The invention will now be illustrated by the following non-limiting examples. All starting materials are commercially available or earlier described in the literature.

### Example 1

### 3-(4-chlorobenzoyl)-6-hydroxy-5-isopropyl-2-methylbenzonitrile

Cuprous cyanide (71 mg, 0.8 mmol) was added to a solution of (4-chlorophenyl)(4-hydroxy-5-isopropyl-2-methylphenyl)methanone (150 mg, 0.4 mmol) in anhydrous DMF (2 mL) under an argon atmosphere. The reaction mixture was refluxed for four hours and cooled down to 70° C. Ferric chloride (260 mg, 1.6 mmol) was added and the mixture stirred for 30 minutes. A 0.2 M solution of NaHSO₄ was added and the aqueous phase extracted with ethyl acetate. Ethyl acetate was evaporated, the material redissolved in DMF (1 ml) and purified by preparative HPLC on a C8-column using a gradient of ammonium acetate buffer/acetonitrile as eluent. Fractions containing the product were pooled, coevaporated twice from water/actonitrile, dissolved in water and then freeze-dried to give the product as a solid (75 mg).

¹H NMR (400 MHz, CHLOROFORM-D) δ ppm 7.71 (d, 2 H), 7.47 (d, 2 H), 7.36 (s, 1 H), 6.61 (s, 1 H), 3.20-3.38 (m, 1 H), 2.47 (s, 3 H), 1.22 (d, 6 H). Mass spectrum: M−H⁺ 312

### Example 2

### (4-chlorophenyl) [4-hydroxy-5-isopropyl-2-methyl-3-(1H-tetrazol-5-yl)phenyl]methanone

Trimethylsilyl azide (52 μL, 0.4 mmol) was added to a solution of 3-(4-chlorobenzoyl)-6-hydroxy-5-isopropyl-2-methylbenzonitrile (40 mg, 0.13 mmol) and n-dibutyltin oxide (10 mg, 0.04 mmol) in toluene (2 mL). The reaction vessel was flushed with argon, sealed and stirred for four days at 100° C. The solvent was evaporated under reduced pressure and the crude mixture purified on silica gel using a gradient of ethyl acetate/heptane. The titled compound was isolated in 1.6 mg (3%) yield as well as 3-(4-chlorobenzoyl)-6-hydroxy-5-isopropyl-2-methylbenzamide (Example 4, see below).

¹H NMR (400 MHz, CHLOROFORM-d) δ ppm 7.80 (d, 2 H), 7.48 (d, 2 H), 7.30 (s, 1 H), 3.38-3.55 (m, 1 H), 2.58 (s, 3 H), 1.22 (d, 3 H). Mass spectrum: M−H⁺ 355

### Example 3

### 3-(4-chlorobenzoyl)-6-hydroxy-5-isopropyl-2-methylbenzamide

3-(4-chlorobenzoyl)-6-hydroxy-5-isopropyl-2-methylbenzamide was isolated in 3.6 mg (8%) yield during the purification of (4-chlorophenyl)[4-hydroxy-5-isopropyl-2-methyl-3-(1H-tetrazol-5-yl)phenyl]methanone in Example 3.

¹H NMR (400 MHz, CHLOROFORM-d) δ ppm 7.74 (d, 2 H), 7.46 (d, 2 H), 7.22 (s, 1 H), 5.94-6.23 (m, 1 H), 3.27-3.45 (m, 1 H), 2.51 (s, 3 H), 1.19 (d, 6 H).

Mass spectrum: M−H⁺ 330

### Example 4

### 1,5-di-tert-butyl-2,4-dimethoxybenzene

NaOH (9 mL, 2M aqueous solution) was added to 4,6-di-tert-butylbenzene-1,3-diol (1 g, 4.5 mmol), tetramethylammonium hydrogen sulfate (0.15 g, 0.45 mmol) and dimethysulfate (1.1 g, 9 mmol) in dichloromethane (30 mL). The solution was refluxed for 30 minutes. The organic phase was separated, extracted with brine, dried over MgSO₄ and solvent evaporated. The crude product was purified by flash chromatography using a 1:4 ratio of ether/heptane as eluent to give the title solid in 0.92 g (82%) yield.

Mass spectrum: m/z M+H⁺ 250

### Example 5

### 3,5-di-tert-butyl-2,6-dimethoxybenzamide

A mixture of 3,5-di-tert-butyl-2,6-dimethoxybenzoic acid (100 mg, 0.34 mmol) thionyl chloride (161 mg, 2.4 mmol) and DMF (one drop) in toluene (1 mL) was heated to 75° C. for 4 hours. The solvent was removed and DCM added. NH₄OH (5 mL, aqueous solution) was added and stirred for 15 minutes. The phases were separated, the aqueous phase was extracted with DCM (3 times), the organic phases combined and washed with water. The organic phase was dried over MgSO₄ and solvent evaporated to give the title product in 88 mg (88%) yield.

¹H NMR (400 MHz, CDCl₃) δ ppm 7.22 (s, 1 H), 3.82 (s, 6 H), 1.33 (s, 18 H).

Mass spectrum: m/z M+H⁺ 294.

### Pharmacology

### In Vitro Model

hGlyRα1 Electrophysiology

Transfected L(tk)⁻ cells stably expressing human GlyRα1 homomers were incubated at 37° C. (5% CO₂) in tissue flasks (Costar) containing Modified Eagle Medium+Earles+L-glutamin (MEM; GibcoBRL) supplemented with 10 % heat-inactivated fetal calf serum, 100 IU/ml Penicillin/Streptomycin (GibcoBRL). Cells were split twice weekly, using mild trypsination. The cells were split and seeded in 50 mm cell culture dishes 24-48 h prior to the experiment.

Glycine receptor-mediated whole-cell currents were recorded under voltage-clamp conditions. Borosilicate glass pipettes (GC150-10, Clark Electromedical Instruments) were used. The cell culture dish was fitted with an inset giving a recording chamber volume of 0.6 ml. The chamber was continuously perfused with extracellular solution (see below) at ˜1.5 15 ml/min. Test compounds were delivered by a DAD-12 superfusion system (Adams & List Associates, Ltd, Westbury, N.Y.; USA). The signals were recorded using an Axopatch 200A amplifier, a Digidata interface and the pClamp software (all from Axon Instruments, Foster City, Calif.). No series resistance compensation was used. All experiments were performed at room temperature.

Extracellular solution contained (in mM): NaCl 137, KCl 5.0, CaCl₂ 1.0, MgCl₂ 1.2, HEPES 10, glucose 10, pH adjusted to 7.4 with NaOH. Intracellular solution contained (in mM): KCl 140, NaCl 3.0, MgCl₂ 1.2, EGTA 1.0, HEPES 10, pH adjusted to 7.2 with KOH.

Glycine (Sigma) stock solution was prepared fresh each day in extracellular solution. The test compounds were dissolved in DMSO to a concentration of 20 MM and diluted in the extracellular solution to the final concentration. The concentration-response curve was obtained by first applying a 40 μM control concentration of glycine for 10 seconds. The lowest concentration of test compound was subsequently applied for 10 seconds alone, then co-applied with 40 μM glycine for 10 seconds. This sequence was repeated with 4 concentrations of test compound on each cell. There was no washout of compound between concentrations.

Raw data was analysed using the pClamp software. The peak currents were measured and normalized to the control glycine current. Concentration-response relationships were plotted using Origin 6.1 (OriginLab® Corporation, Northampton, Mass.).

Typical IC₅₀ values for the compounds of the present invention are in the range of about 0.1 to about 1,000,000 nM. Other values for IC₅₀ are in the range of about 1 to about 100,000 nM. Further values for IC₅₀ are in the range of about 10 nM to about 30,000 nM

### In Vivo Model

### Freund's Complete Adjuvant (FCA) Induced Arthritis in Rat

### Animals

Male Sprague Dawley rats (B&K Universal AB, Uppsala, Sweden) weighing 150 to 300 g at the time of FCA injection are being used. Rats are held up to 6 in transparent Macrolon® IV cages with wood shavings as bedding. Holding and study areas have automatic control of the light cycle (12:12 hr), the temperature (21±2° C.) and the humidity (40 to 80%).

### Experimental Procedure

Under isoflurane anesthesia, 40 μl of FCA (1 mg/mL) is injected into the left tibio-tarsal (ankle) joint from the dorsal side of the rats. The injection causes a localized inflammation and the animals display decreased weight bearing on and guarding of the limb. The animals are allowed to recover in their home cage for 48 hours following the injection of FCA before any experiment is performed. Forty-eight hours after induction of arthritis and at measurement times depending on the kinetics of the test compound, the rats are placed in a Plexiglas chamber and videotaped for 5 min from underneath. Subsequently, the weight the rats were willing to put on the injected paw are scored as 0: normal paw position, 1: the paw is used during walking, but the toes are kept together, 2: pronounced limping, 3: the paw does not contact the floor.

### Administration of the Substance

Rats are injected orally, subcutaneously or intraperitonealy depending on kinetic profile of the test substance. The time between administration and videotaping is also dependant on the kinetics of the test compound.

### Neuropathic Pain Model—Modified Chung Model

### Animals

Male Sprague-Dawley (Hsd:SD) rats (Charles River, St Constant, Canada) weighing approximately 100-150 g are ordered for surgery. Rats are housed in groups of 7-9 in a temperature controlled room (22±1.5° C., 30-80% humidity, 12 h light/dark cycle). Rats are acclimatized in the animal facility for at least one-day prior to use. Experiments are performed during the light phase of the cycle, rooms are illuminated at 300 lux intensity. Animals have food and water ad libitum.

### Experimental Procedures—Modified Spinal Nerve Ligation Model (Also Called Modified SNL or Modified Chung Model) (Chung et al. 2004)

Under ketamine and xylazine anesthesia, a dorsal mid-line incision are made approximately from the lower lumbar (L3) level to sacral (S2) level allowing exposure of the muscles. The left paraspinal muscles are isolated and removed from the L4 spinous level to the sacrum S1 level. The bone, L6 transverses process, is then removed to allow easy access to the L5 spinal nerve. The left L5 and L6 spinal nerves are carefully isolated and tightly ligated with 4-0 silk threads whereas L4 is “tickled” about 10 times using glass hook. The incision is closed in layers using an appropriate suture material. Rats are allowed to recuperate until post-operative day 10 at which time testing can begin.

### Test Procedure

Rats are placed on a grid floor, and are covered by a reversed small animal cage. In order to determine the rat's threshold (measured in g) to a tactile mechanical stimulus baseline measurements are determined by touching the treated paw with a series of monofilaments of incremental stiffness in the “up/down” method (Chaplan et al. (1994)).

After determination of the rat's threshold to the tactile mechanical stimulus, rats are randomized in homogeneous groups before experiments are started. Rats having mechanical threshold higher than 5 g are exclude from the study.

### Administration of the Substance

Rats are injected orally, subcutaneously or intraperitonealy depending on kinetic profile of the test substance. The time between administration and videotaping is also dependant on the kinetics of the test compound.

### List of Abbreviations

HEPES 4-(2-Hydroxyethyl)piperazine-1-ethanesulfonic acid
EGTA Ethylene glycol-bis(2-aminoethylether)-N,N,N,N′-tetraacetic acid

1-30. (canceled)

31. A compound of formula I, or a pharmaceutically acceptable salt thereof 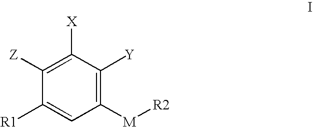
wherein:
X is selected from hydrogen, halo, —CN, —CONH₂, —CON(C₁₋₆alkyl)H, —CON(C₁₋₆alkyl)₂ or a heterocyclic group;
R1 is selected from C₁₋₆alkyl or C₃₋₆cycloalkyl;
wherein
Y is —C₁₋₆alkyl;
Z is —OH,
M is selected from C(O), CH(OR^{a}), N(R^{a}), or S(O)ᵣ, wherein R^{a} is hydrogen or C₁₋₆alkyl and r is 0, 1 or 2,
R2 is selected from C₁₋₆alkyl, C₃₋₆cycloalkyl, heterocycloalkyl, aryl, alkylaryl, or heteroaryl,
R2 is substituted with halo, —NO₂, —CN, —OH, —CF₃, —OCF₃, —NH₂, or —CONH₂, or
Y is —C₁₋₆alkoxy;
Z is —C₁₋₆alkoxy,
M is bond,
R2 is selected from C₁₋₆alkyl, C₃₋₆cycloalkyl, heterocycloalkyl, aryl, alkylaryl, or heteroaryl.

32. A compound of claim 31 wherein X is selected from hydrogen, halo, —CN, —CONH₂, or heterocyclic groups.

33. A compound of claim 31 wherein X is selected from hydrogen, —Br, —CN, —CONH₂, or tetrazolyl.

34. A compounds of claim 31 wherein: R1 is C₃₋₄alkyl.

35. A compounds of claim 31 wherein:
Z is —OH,
M is C(O),
R2 is selected from C1-6alkyl, C₃₋₆cycloalkyl, heterocycloalkyl, aryl, alkylaryl, or heteroaryl,
R2 is substituted with halo, —NO₂, —CN, —OH, —CF₃, —OCF₃, —NH₂, or —CONH₂, and
Y is —C1-6alkyl.

36. A compound of claim 31 wherein R2 is aryl.

37. A compound of claim 31 wherein R2 is selected from phenyl, naphthyl, cyclohexyl, methylbenzyl, or quinoxalinyl.

38. A compound according to claim 31 wherein
Z is —OH,
M is selected from C(O), CH(OR^{a}), N(R^{a}), or S(O)ᵣ, wherein R^{a} is hydrogen or C1-6alkyl and r is 0, 1 or 2,
R2 is selected from C1-6alkyl, C₃₋₆cycloalkyl, heterocycloalkyl, aryl, alkylaryl, or heteroaryl,
R2 is substituted with halo,
and Y is —C1-6alkyl.

39. A compound according to claim 38 wherein R2 is substituted with chloro.

40. A compound according to claim 31 wherein X is selected from hydrogen, —Br, —CN, —CONH₂, or tetrazolyl;
Y is —CH₃;
R1 is C₃₋₄alkyl;
M is C(O);
R2 is selected from phenyl, naphthyl, cyclohexyl, methylbenzyl, or quinoxalinyl,
R2 is substituted with chloro; and
Z is —OH.

41. A compound according to claim 31 wherein X is selected from hydrogen, —Br, —CN, —CONH₂, or tetrazolyl;
Y is —OCH₃;
R1 is C₃₋₄alkyl;
M is a bond;
R2 is selected from phenyl, naphthyl, cyclohexyl, methylbenzyl, or quinoxalinyl; and
Z is —OCH₃.

42. A compound selected from the group consisting of
(4-chlorophenyl) [4-hydroxy-5-isopropyl-2-methyl-3-(1H-tetrazol-5-yl)phenyl]methanone,
(4-hydroxy-5-isopropyl-2-methylphenyl)(quinoxalin-2-yl)methanone,
3-(4-chlorobenzoyl)-6-hydroxy-5-isopropyl-2-methylbenzamide,
3-(4-chlorobenzoyl)-6-hydroxy-5-isopropyl-2-methylbenzonitrile,
3,5-di-tert-butyl-2,6-dimethoxybenzamide, and
1,5-di-tert-butyl-2,4-dimethoxybenzene.

43. A pharmaceutical composition comprising as active ingredient a therapeutically effective amount of a compound according to formula I, or pharmaceutically acceptable salts thereof 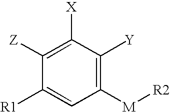
wherein
X is selected from hydrogen, halo, —CN, —CONH₂, —CON(C₁₋₆alkyl)H, —CON(C₁₋₆alkyl)₂ and heterocyclic groups;
R1 is selected from C₁₋₆alkyl and C₃₋₆cycloalkyl;
wherein
Y is —C1-6alkyl;
Z is —OH,
M is selected from C(O), CH(OR^{a}), N(R^{a}), or S(O)ᵣ, wherein R^{a} is hydrogen or C₁₋₆alkyl and r is 0, 1 or 2,
R2 is selected from C₁₋₆alkyl, C₃₋₆cycloalkyl, heterocycloalkyl, aryl, alkylaryl, or heteroaryl,
R2 is substituted with halo, —NO₂, —CN, —OH, —CF₃, —OCF₃, —NH₂, or —CONH₂; or
Y is —C1-6alkoxy;
Z is —C1-6alkoxy,
M is a bond,
R2 is selected from C₁₋₆alkyl, C₃₋₆cycloalkyl, heterocycloalkyl, aryl, alkylaryl, or heteroaryl, in association with one or more pharmaceutically acceptable diluents, excipients or inert carriers.

44. A pharmaceutical composition of claim 43 wherein in said compound, X is selected from hydrogen, halo, —CN, —CONH₂, or a heterocyclic group.

45. A pharmaceutical composition of claim 43 wherein in said compound, X is selected from hydrogen, —Br, —CN, —CONH₂, or tetrazolyl.

46. A pharmaceutical composition of claim 43 wherein in said compound, R1 is C₃₋₄alkyl.

47. A pharmaceutical composition of claim 43 wherein in said compound,
Z is —OH,
M is C(O),
R2 is selected from C₁₋₆alkyl, C₃₋₆cycloalkyl, heterocycloalkyl, aryl, alkylaryl, or heteroaryl,
R2 is substituted with halo, —NO₂, —CN, —OH, —CF₃, —OCF₃, —NH₂, and/or —CONH₂, and
Y is —C1-6alkyl.

48. A pharmaceutical composition of claim 43 wherein in said compound, R2 is aryl.

49. A pharmaceutical composition of claim 43 wherein in said compound, R2 is selected from phenyl, naphthyl, cyclohexyl, methylbenzyl, or quinoxalinyl.

50. A pharmaceutical composition of claim 43 wherein in said compound,
Z is —OH,
M is selected from C(O), CH(OR^{a}), N(R^{a}), or S(O)ᵣ, wherein R^{a} is hydrogen or C₁₋₆alkyl and r is 0, 1 or 2,
R2 is selected from C₁₋₆alkyl, C₃₋₆cycloalkyl, heterocycloalkyl, aryl, alkylaryl, or heteroaryl,
R2 is substituted with halo, and
Y is —C1-6alkyl.

51. A pharmaceutical composition of claim 50 wherein in said compound, R2 is substituted with chloro.

52. A pharmaceutical composition of claim 43 wherein in said compound, X is selected from hydrogen, —Br, —CN, —CONH₂, or tetrazolyl;
Y is —CH₃;
R1 is C₃₋₄alkyl;
M is C(O);
R2 is selected from phenyl, naphthyl, cyclohexyl, methylbenzyl, or quinoxalinyl,
R2 is substituted with chloro; and
Z is —OH.

53. A pharmaceutical composition of claim 43 wherein in said compound, X is selected from hydrogen, —Br, —CN, —CONH₂, or tetrazolyl;
Y is —OCH₃;
R1 is C₃₋₄alkyl;
M is a bond;
R2 is selected from phenyl, naphthyl, cyclohexyl, methylbenzyl, or quinoxalinyl; and
Z is —OCH₃.

54. A method of treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain;
pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus;
ophthalmological disorders such as retinopathies, diabetic retinopathies or glaucoma; and/or psychiatric disorders, such as alcoholism, drug addiction and psychosis, comprising administering to a subject in need of such treatment, a therapeutically effective amount of the compound according to claim 1.

55. A method according to claim 24, treating acute or chronic neuropathic pain.

56. A process for preparing a compound of formula I, wherein X, Y, Z, R1, and R2 are, unless specified otherwise, defined as in formula I, comprising:
a) reacting a compound of formula (II) 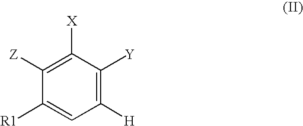
i) with a compound of formula (III) in a suitable solvent, wherein W is a halogen or a suitable leaving group 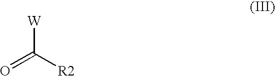
or
ii) with a compound of formula (IV) wherein W is a halogen, and n is 0, 1, or 2, in a suitable solvent, optionally followed by treatment with an oxidation reagent in case n is 0 or 1, 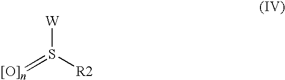
or
b) reacting a compound of formula (V), wherein Hal is a halogen or a sulfonyloxy group, and X is a non-protic or protected functional group, 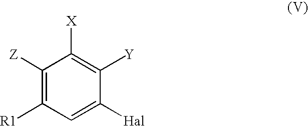
i) with an organometallic reagent of formula (VI), wherein Met is a suitable metallic group, or an organoboron reagent, 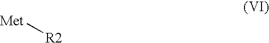
in the presence of a carbon monoxide or dry nitrogen atmosphere, and in the presence of a metallic catalyst,
or
ii) with an amine of formula (VII), 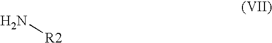
in the presence of a metallic catalyst, and in the presence of a suitable inert solvent or diluent, or
iii) with a mercaptan of formula (X), 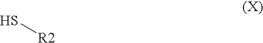
in the presence of a metallic catalyst, and in the presence of a suitable base, optionally followed by oxidation by treatment with an oxidation reagent to give a sulfoxide or sulfone,
c) reacting a compound of formula (VIII), 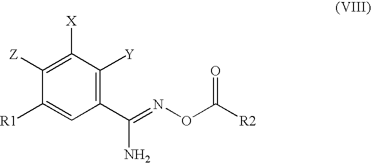
by heating in a suitable solvent between 30° C. and reflux, or
d) reaction of a compound of formula (XII), wherein Hal is a halogen or a sulfonyloxy group, 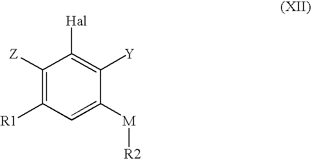
with a suitable cyanide nucleophile in a suitable solvent at a temperature between 50° C. and reflux under an inert atmosphere or
e) reacting a compound of formula (XIII) 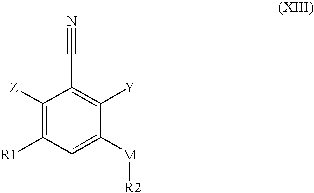
with a suitable azide reagent, in the presence of a catalyst in a suitable solvent at a temperature between 50° C. and reflux under an inert atmosphere, or
f) reacting an acid of formula (XVI), 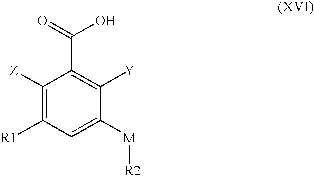
with a suitable halogenation reagent, in a suitable solvent at a temperature between ambient and reflux followed by treatment with an amine or ammonia solution,
i) converting a compound of the formula I into another compound of the formula I; and/or
ii) removing any protecting groups; and/or
iii) forming a pharmaceutically acceptable salt.
